(19) United States
(12) Patent Application Publication
(10) Pub. No.: US 2004/0200479 A1
(43) Pub. Date: Oct. 14, 2004

(54) Laryngeal mask airway

(76) Inventor: Ti-Li Chang, Taichung Hsien (TW)

Correspondence Address: HERSHKOVITZ & ASSOCIATES, 1725 I STREET NW, SUITE 300, WASHINGTON, DC, 20006, US

(21) Appl. No.: 10/809,391

(22) Filed: Mar. 26, 2004

(30) Foreign Application Priority Data
Apr. 10, 2003 (TW) 092205583

(51) Int. Cl. A61M 16/00

(52) U.S. Cl. 128/207.14

(57) ABSTRACT

A laryngeal mask airway has a dual-airway, a back plate with a smooth extended tongue and a laryngeal mask with a top surface. The back plate is integrally formed with the top surface of the laryngeal mask. The dual-airway communicates with the back plate. The dual-airway further has a secondary tube for injecting air into the bladder. Because the extended tongue of back plate is integrally formed on the laryngeal mask, laryngeal mask airways in accordance with the present invention are easy and have a guide to insert into the larynx without the tip folding over.

### BACKGROUND OF THE INVENTION

1. Field of Invention

The present invention relates to a laryngeal mask airway, more particular to a laryngeal mask airway that does not fold over at the tip.

2. Description of the Related Art

With reference to FIGS. 6 and 7, a conventional laryngeal mask airway has a laryngeal mask (70), an inflation tube (74) and an airway tube (60).

The laryngeal mask (70) has a body (not numbered), a front end (not numbered), a rear end (not numbered), a curved bottom face (not numbered), a top face (not numbered), a bladder (72) and multiple ventilation holes (71). The bladder (72) is integrally mounted around the body of the laryngeal mask (70). The multiple ventilation holes (71) are defined through the body from the curved bottom face to the top face.

The inflation tube (74) is connected to and communicates with the rear end of the laryngeal mask (70) and has a diameter (not numbered), a front end (not numbered), an inflation balloon (76), a valve (77) and a rear end (not numbered). The front end of the inflation tube (74) is connected to the bladder (72) of the laryngeal mask (70) near the rear end of the laryngeal mask (70). The inflation balloon (76) is attached to the rear end of the laryngeal inflation tube (74), and the valve (77) is connected to the inflation balloon (76).

The airway tube (60) is a large-bore tube made of resilient plastic material, is attached to the top face of the laryngeal mask (70) near the rear end and communicates with the ventilation holes (71). The airway tube (60) has a front end (not numbered) and a rear end (not numbered).

When the laryngeal mask is used to treat a patient, air inside the bladder (72) must be removed first. Then the laryngeal mask (70) is inserted into the patient's mouth against hard plate, soft plate to esophagus until the ventilation holes (71) communicated with the larynx of the patient. With the bladder (72) around the opening to the larynx, the bladder (72) would be inflated air by syringe through the valve to form a seal around the larynx. Anesthetic gas can be injected into the lungs of the patient through the airway tube (60) without any gas leakage.

However, the conventional laryngeal mask airway has the following disadvantages:

1. The inflation tube (74) and the airway tube (60) are attached separately to the laryngeal mask. When the laryngeal mask airway is inserted into a patient's mouth, the two separate tubes will make the process more difficult.

2. When the air inside the bladder (72) is removed, the bladder (72) is flat. The flat and soft bladder (72) folds over too easily when approaching the larynx, which may keep the bladder (72) from successfully sealing the larynx.

3. The laryngeal mask (70) is not easy to insert because the laryngeal mask (70) does not have a leading element. Passing the laryngeal mask (70) through the hard palate and soft palate to the larynx is difficult, and the person inserting the laryngeal mask (70) often needs to try many times before successfully inserting the laryngeal mask (70). This difficulty causes considerable discomfort to an already ill person and may cause the patient to vomit. The present invention provides a laryngeal mask airway to mitigate or obviate the aforementioned problems.

### SUMMARY OF THE INVENTION

The primary object of the present invention is to provide a laryngeal mask airway that is convenient and efficient to use.

To achieve the object, the laryngeal mask airway has a dual-airway, a back plate with a smooth extended tongue and a laryngeal mask with a top surface. The back plate is integrally formed with the top surface of the laryngeal mask. The dual-airway communicates with the back plate. The dual-airway further has a secondary tube for injecting air into the bladder.

Because the extended tongue of back plate is integrally formed on the laryngeal mask, the laryngeal mask airways in accordance with the present invention are easy to insert into the larynx without the folding over on back plate.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of a laryngeal mask airway in accordance with the present invention;

FIG. 2 is a cross sectional side plan view of the laryngeal mask airway in FIG. 1;

FIG. 3 is a side plan view of the laryngeal mask airway in FIG. 1;

FIG. 4 is an operational perspective view of the laryngeal mask airway in FIG. 1 passing through the mouth;

FIG. 5 is an operational perspective view of the laryngeal mask airway in FIG. 1 covering the larynx;

FIG. 6 is a perspective view of a conventional laryngeal mask airway in accordance with the prior art; and

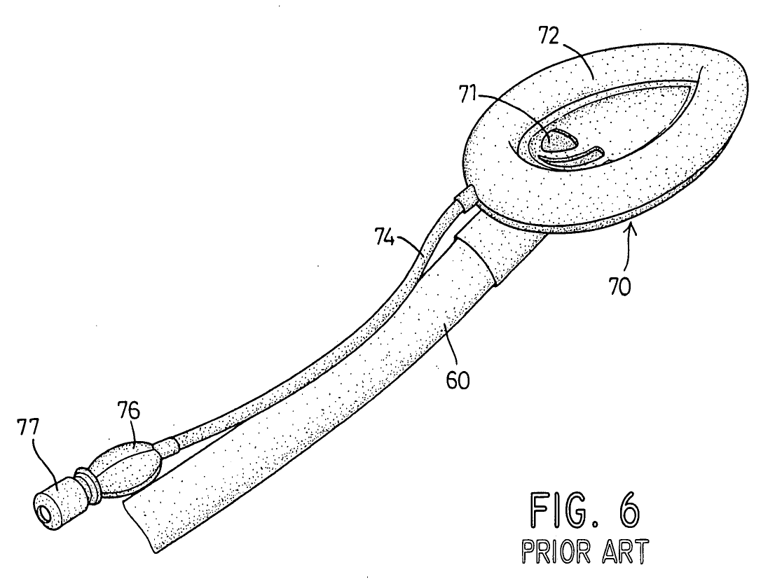

FIG. 7 is an operational side plan view of the conventional laryngeal mask airway in FIG. 6.

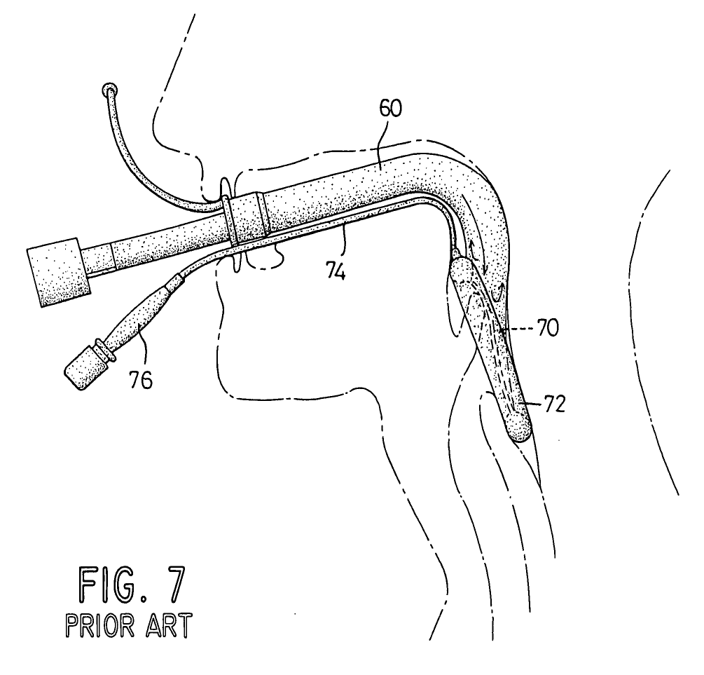

### DETAILED DESCRIPTION OF THE INVENTION

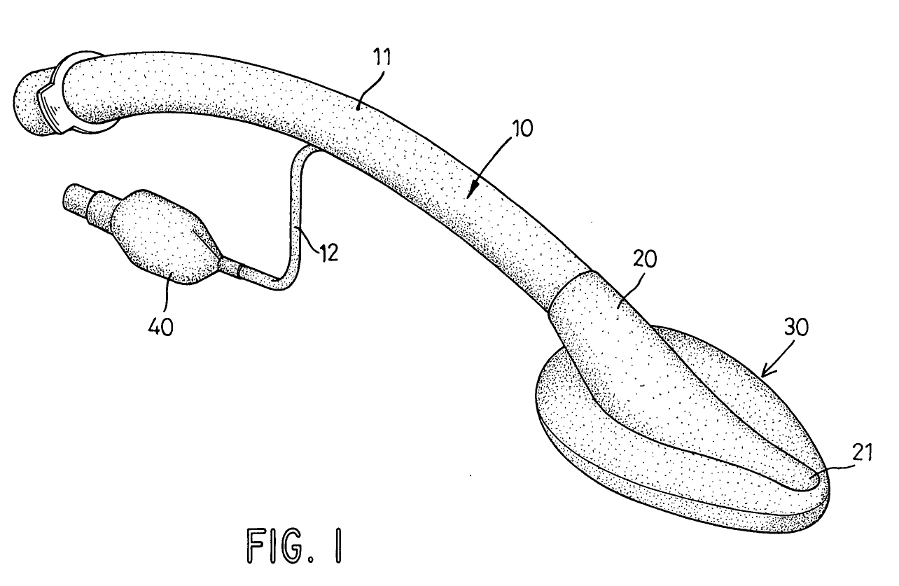

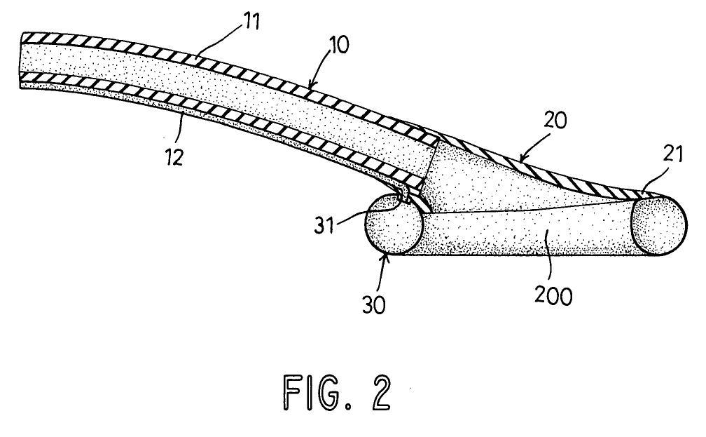

With reference to FIGS. 1 to 3, a laryngeal mask airway has a dual-airway (10), a back plate (20) and a laryngeal mask (30).

The dual-airway (10) is curved and has a rear end (not numbered), a front end (not numbered), a primary tube (11), a secondary tube (12) and an inflation balloon (40). The primary tube (11) has a front end (not numbered), a rear end (not numbered) and a bottom surface (not numbered). The rear end of the primary tube (11) is used for inputting medical gas. The secondary tube (12) has a rear end (not numbered) and a front end (not numbered) and is mounted on the bottom surface of the primary tube (11) at the front end. The inflation balloon (40) is mounted on the secondary tube (12) near the rear end of the secondary tube (12) and communicates with the secondary tube (12).

The back plate (20) is connected to the front end of the primary tube (11), is horn-shaped and has a rear end (not numbered), a front end (not numbered), a side edge (200) and an extended tongue (21). The rear end of the back plate (20) is connected to the front end of the dual-airway (10). The extended tongue (21) is integrally formed with and extends out from the front end of the back plate (20). The extended tongue (21) of the back plate (20) is raised about seven degrees (7°) relative to the front end of the back plate (20).

The laryngeal mask (30) is connected to the side edge (200) of the back plate (20) and has a connecting hole (31). The connecting hole (31) is defined in the laryngeal mask (30) and communicates with the front end of the secondary tube (12) to inflate the bladder.

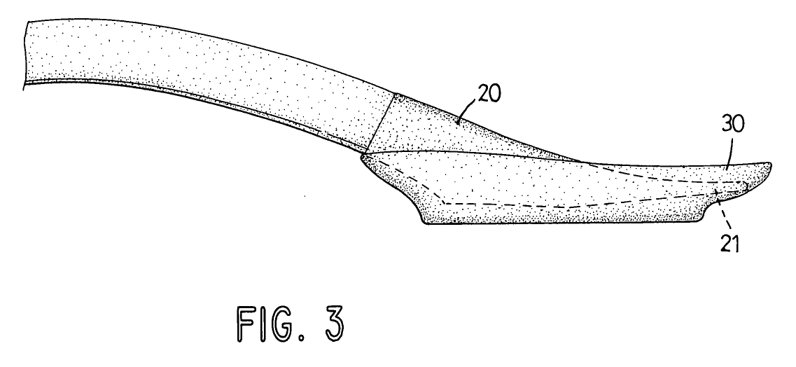

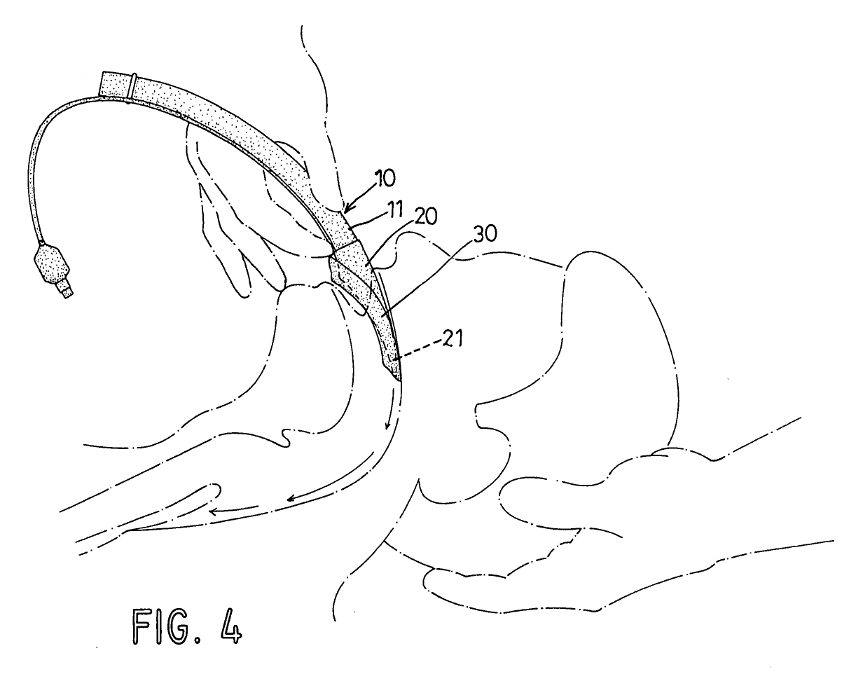

With reference to FIGS. 3 and 4, the laryngeal mask (30) becomes like a boat when the air inside the laryngeal mask (30) is removed. The rising tip (21) of the back plate (20) mounted on the laryngeal mask (30) will make the laryngeal mask (30) insert into the throat easily and also press the laryngeal mask (30) to guide and keep the laryngeal mask (30) from folding over easily. The dual-airway (10) with the boat-shaped laryngeal mask (30) is fed forward along the hard palate until definite resistance is felt. The tip (21) of the mask is then located in the hypopharynx, and the opening of the mask will face the larynx of the patient.

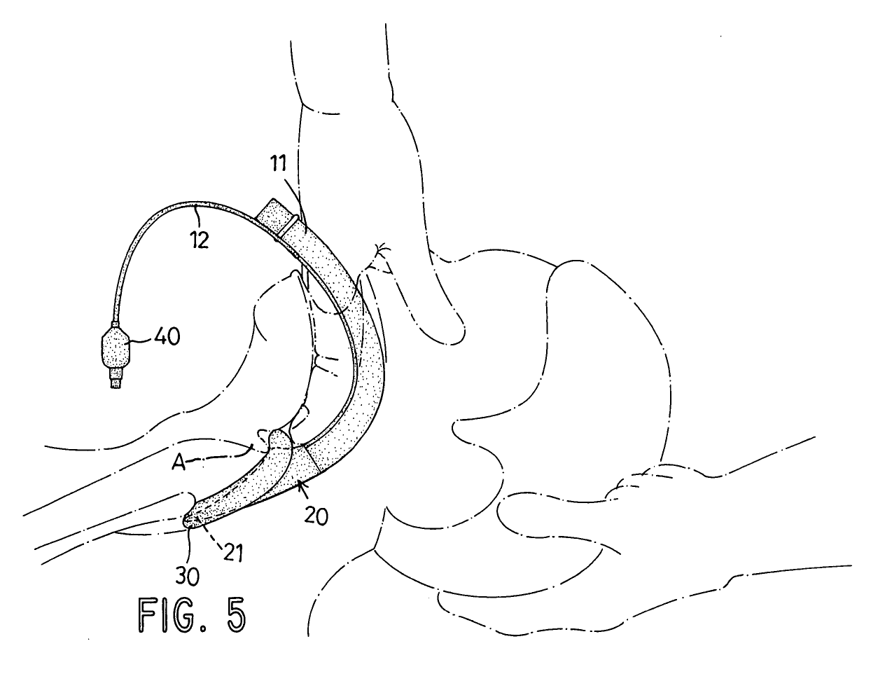

With reference to FIG. 5, the inflation syringe is used inject air into the secondary tube (12) to make the laryngeal mask (30) fill with air when the tip of laryngeal mask (30) arrives the esophagus. The laryngeal mask (30) is then inflated with proper air volume to form a proper seal around the opening to the larynx.

The laryngeal mask airway in accordance with the present invention has the following advantages.

1. When the air inside the laryngeal mask (30) is removed, the raised tip (21) will press the laryngeal mask (30) to guide and easily insert the laryngeal mask airway into a patient, and the front end of the inflatable body will not easily fold over.

2. The secondary tube (12) is integrally mounted on the dual-airway (10), and such assembly will make the laryngeal mask airway easy to insert into a patient.

The invention may be varied in many ways by a person skilled in the art. Such variations are not to be regarded as a departure from the spirit and scope of the invention, and all such modifications are intended to be included within the scope of the following claims.

What is claimed is:

1. A laryngeal mask airway having
a dual-airway having
a primary tube used for inputting medical gas and having a bottom surface and a front end,
a secondary tube integrally mounted on the bottom surface of the primary tube and having
a rear end and a front end, and
an inflation balloon mounted on the secondary tube near the rear end of the secondary tube and communicates with the secondary tube,
a back plate connected to the front end of the primary tube and having
a rear end connected to the front end of the primary tube,
a front end,
a side edge, and
a tongue integrally formed with and extending out from the front end of the back plate, and raising at a degree relative to the front end of the cover, and
a laryngeal mask connected to the side edge of the back plate and having
an opening defined in the laryngeal mask and communicating with the front end of the secondary tube.

2. The laryngeal mask airway as claimed in claim 1, wherein the extended tongue of the back plate rises at about 7 degrees.
